(19) United States
(12) Patent Application Publication
(10) Pub. No.: US 2026/0191467 A1
(43) Pub. Date: Jul. 9, 2026

(54) SENSING SYSTEM FOR BIOMETRIC MEASUREMENTS

(71) Applicant: Tacta Systems Inc., Palo Alto, CA (US)

(72) Inventors: Nahid Harjee, Sunnyvale, CA (US); David Bibl, Scotts Valley, CA (US)

(21) Appl. No.: 19/013,845

(22) Filed: Jan. 8, 2025

(51) Int. Cl. A61B 5/00; A61B 5/0205; A61B 5/11

(52) U.S. Cl. CPC A61B 5/6806; A61B 5/0205; A61B 5/4082; A61B 5/4088; A61B 5/7246; A61B 5/746; A61B 5/1101; A61B 5/112; A61B 2560/0209; A61B 2562/028; A61B 2562/06

(57) ABSTRACT

A system may include a wearable article to be worn by a user. For example, the wearable article may include a glove with digits having digit sections, or a sleeve to be worn on an arm or leg. A sensor array may be coupled to the wearable article. The sensor array may include sensors that are submillimeter in at least one in-plane dimension and are distributed at multiple locations of a sensing area of the wearable article. The system may also include a processor configured to: obtain a biometric measurement from the sensor array, the biometric measurement including a spatial map of sensed conditions from the sensors at the multiple locations; and generate an output based on comparing the spatial map to a baseline map of reference values corresponding to the multiple locations. Other aspects are also described and claimed.

### BACKGROUND

### Field

This disclosure relates generally to sensing systems and, more specifically, to a sensing system for biometric measurements. Other aspects are also described.

### Background Information

A sensor may refer to a device that produces an output signal for detecting a physical phenomenon. A group of sensors may form a sensor array which may be used for collecting information about an environment. Sensors of a sensor array may be arranged in a certain geometric configuration or pattern. Sensor arrays may enable collecting information over a greater area than a single sensor.

In operation, a sensor of a sensor array can generate an output signal indicating detection of a physical phenomenon. For example, a piezoelectric sensor can utilize the piezoelectric effect to detect changes in pressure, acceleration, strain, or force by converting such detections to electrical charge. In another example, a capacitive sensor can utilize change in capacitance to detect an object in proximity that may be conductive or may have a dielectric constant that is different from air.

### SUMMARY

Implementations of this disclosure include integrating a tactile sensing array with a wearable article to form a wearable sensing device (or simply sensing device) to be worn by a user. The sensing device can utilize microsensors in the sensing array, e.g., sensors that are submillimeter in at least one in-plane dimension (e.g., a dimension of its footprint), to obtain a high spatial resolution biometric measurements that are less than 2 millimeters (mm) apart, and in some cases, less than 1 mm apart. Further, the biometric measurements may be conveniently obtained over extended periods of time, e.g., periodically during a multi-hour shift, multiple days of a week, or multiple weeks of a month. The sensing device can operate in a system to sense, evaluate, and/or monitor biometric measurements of the user or another individual and to provide outputs based on the sensing, evaluating, and/or monitoring.

Some implementations may include a system for biometric measurements. The system may include a wearable article to be worn by a user. A sensor array may be coupled to the wearable article. The sensor array may include sensors that are submillimeter in at least one in-plane dimension and are distributed at multiple locations of a sensing area of the wearable article. The system may also include a processor configured to: obtain a biometric measurement from the sensor array, the biometric measurement including a spatial map of sensed conditions from the sensors at the multiple locations; and generate an output based on comparing the spatial map to a baseline map of reference values corresponding to the multiple locations.

Some implementations may include a method for biometric measurements. The method may include: obtaining a biometric measurement from a sensing device comprising a sensor array coupled to a wearable article, wherein the sensor array includes sensors that are submillimeter in at least one in-plane dimension and are distributed at multiple locations of a sensing area of the wearable article, and wherein the biometric measurement includes a spatial map of sensed conditions from the sensors at the multiple locations; and generating an output based on comparing the spatial map to a baseline map of reference values corresponding to the multiple locations. Other aspects are also described and claimed.

The above summary does not include an exhaustive list of all aspects of the present disclosure. It is contemplated that the disclosure includes all systems and methods that can be practiced from all suitable combinations of the various aspects summarized above, as well as those disclosed in the Detailed Description below and particularly pointed out in the Claims section. Such combinations may have particular advantages not specifically recited in the above summary.

### BRIEF DESCRIPTION OF THE DRAWINGS

Several aspects of the disclosure herein are illustrated by way of example and not by way of limitation in the figures of the accompanying drawings in which like references indicate similar elements. It should be noted that references to “an” or “one” aspect in this disclosure are not necessarily to the same aspect, and they mean at least one. Also, in the interest of conciseness and reducing the total number of figures, a given figure may be used to illustrate the features of more than one aspect of the disclosure, and not all elements in the figure may be required for a given aspect.

FIG. 1 is an example of a front side of a sensing device.

FIG. 2 is an example of a back side of a sensing device.

FIG. 3 is an example of a cross section of a sensing device worn by a user.

FIG. 4 is an example of a sensor array coupled to a wearable article.

FIG. 5A is an example of a spatial map of sensed conditions.

FIG. 5B is an example of a baseline map of sensed conditions.

FIG. 6 is an example of a user wearing multiple sensing devices.

FIG. 7 is an example of a system including a sensing device.

FIG. 8 is a block diagram of an example internal configuration of a computing device for utilizing a sensing device.

FIG. 9 is an example of a process for utilizing a sensing device.

FIG. 10 is an example of a first process for determining peripheral neuropathy.

FIG. 11 is an example of a second process for determining peripheral neuropathy.

FIG. 12 is an example of a process for determining Parkinson's disease.

FIG. 13 is an example of a process for determining dementia.

FIG. 14 is an example of a first process for determining a blood clot.

FIG. 15 is an example of a second process for determining a blood clot.

FIG. 16 is an example of a process for determining a tumor.

FIG. 17 is an example of a process for monitoring safety or ergonomics.

### DETAILED DESCRIPTION

A biometric measurement may refer to data obtained from an individual's physical or behavioral traits, used to uniquely identify or verify their identity. In various applications, biometric measurements may include fingerprints, facial recognition, iris or retina scans, and/or voice patterns. These measurements can analyze distinguishing physical characteristics (e.g., fingerprint ridges), which may remain consistent over time and may be unique to each person.

In a healthcare environment, biometric measurements can include physiological indicators such as heart rate, blood oxygen levels, and gait analysis. These indicators can help in monitoring health conditions, diagnosing health concerns, and/or creating health profiles of individuals. However, many systems today are limited by a low spatial resolution that is often inadequate for evaluating biometric measurements over large and diverse areas, such as fingers, hands, arms, legs, etc. For systems that do provide a high spatial resolution, such as an magnetic resonance imaging (MRI), ultrasound, or x-ray system, they are often part of expensive, heavy equipment that is difficult for an individual to conveniently use. What is needed is a system for obtaining, evaluating, and/or monitoring biometric measurements for individuals that is convenient, accurate, and cost-effective.

Implementations of this disclosure may address problems like these by integrating a tactile sensing array with a wearable article to form a wearable sensing device (or simply sensing device) to be worn by a user. The sensing device can utilize microsensors in the sensing array, e.g., sensors that are submillimeter in at least one in-plane dimension (e.g., a dimension of its footprint), to obtain a high spatial resolution biometric measurements that are less than 2 millimeters (mm) apart, and in some cases, less than 1 mm apart. Further, the biometric measurements may be conveniently obtained over extended periods of time, e.g., periodically during a multi-hour shift, multiple days of a week, or multiple weeks of a month. The sensing device can operate in a system to sense, evaluate, and/or monitor biometric measurements of the user or another individual and to provide outputs based on the sensing, evaluating, and/or monitoring.

Some implementations may include a system including a wearable article to be worn by a user. For example, the wearable article may include a glove with digits having digit sections (e.g., a thumb and fingers with a thumb tip and fingertips, respectively, and sections between joints), or a sleeve to be worn on an arm or leg (e.g., a compression garment worn on a limb). A sensor array may be coupled to the wearable article to form the sensing device. The sensor array may include sensors that are submillimeter in at least one in-plane dimension and are distributed at multiple locations of a sensing area of the wearable article. For example, the sensors may be microsensors arranged in sensing areas of the wearable article, such as digit sections (a fingertip, thumb tip, or between joints), a palm, wrist, arm, leg, waist, etc.

The system may include a processor executing instructions stored in memory. The processor and memory may be part of the sensing device (e.g., a fully independent operation of the sensing device) and/or may be in communication with the sensing device (e.g., integrated with another computing system, such as a mobile device (smartphone, smartwatch, tablet, etc.), server, or cloud based computing system). The processor can utilize readout circuitry of the sensing device to obtain a biometric measurement from the sensor array. The biometric measurement may include a spatial map of sensed conditions from the sensors at the multiple locations of a sensing area of the sensing area. The processor can then generate an output (feedback) based on comparing the spatial map to a baseline map of reference values corresponding to the multiple locations. As a result, the system can obtain, evaluate, and/or monitor biometric measurements for a user or another individual in a way that is convenient, accurate, and cost-effective.

In some implementations, the sensing device can obtain absolute orientation and position of movement; relative orientation of sections with respect to another section (e.g., digits or digit sections relative to a wrist portion); pressure or force distributions from sensor arrays mounted on multiple sides of the sensing device; and/or proximity, temperature, imaging, and/or conductivity data from sensor arrays mounted on the sides (interior and/or exterior). The sensing device may include the wearable article (e.g., a textile, such as a fabric or elastomer, formed as a wearable), flexible circuits with arrays of microsensors, including force, temperature, proximity, image, and/or conductivity sensors, coupled to the wearable article with an adhesive; assembled motion sensors (e.g., a multi-axis inertial measurement unit (IMU)) and/or optical markers for tracking of the sections; a processor of the sensing device can operate as a local host controller; a power source of the sensing device may include a battery and/or charging port or connector; a communications device of the sensing device may enable wireless communications with a mobile device, server, or cloud based computing system; flexible encapsulation of the sensing device may be applied to the sensors and sensor arrays (e.g., to protect the microsensors, embedded in arrays of the sensing device, from environmental conditions); and/or strain-relief patterns of the sensing device may enable the flexible circuits to conform and bend with the flexing of each joint and movement of the user.

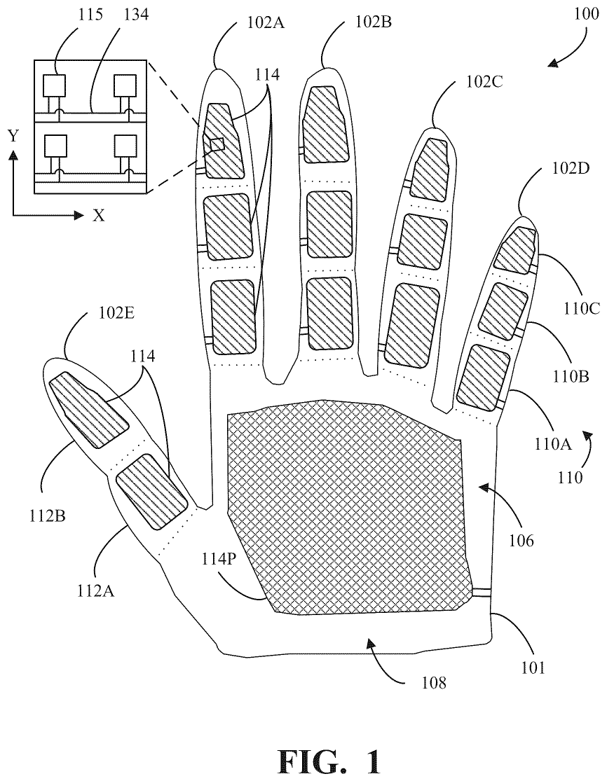

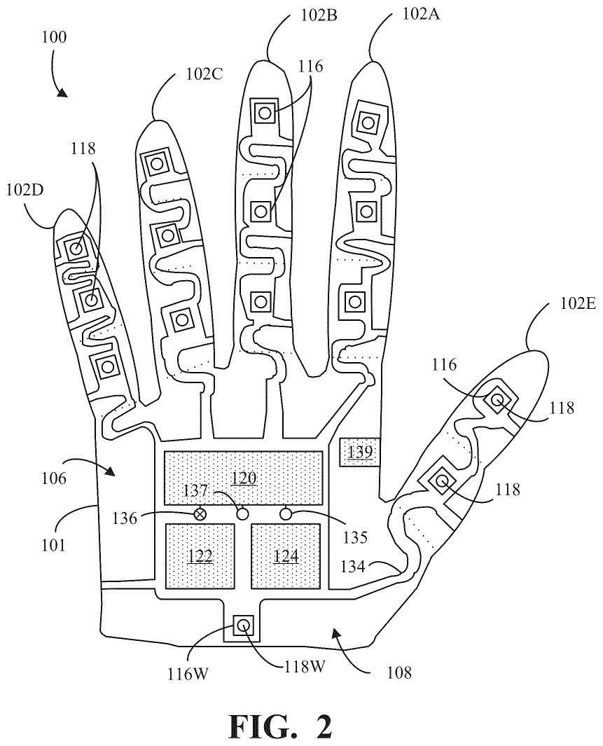

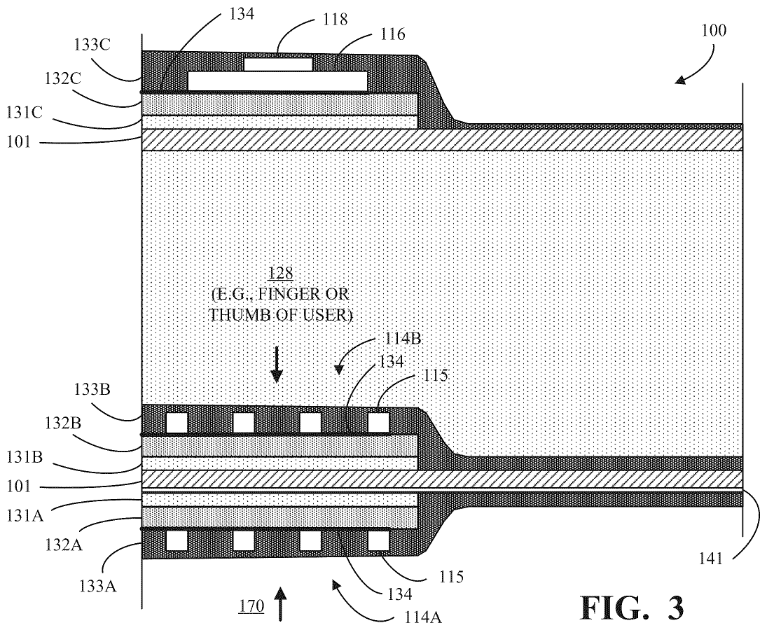

FIG. 1 is an example of a front side of a sensing device 100, shown as a palmar side of a left sensing glove. FIG. 2 is an example of a back side of the sensing device 100, shown as a dorsal side of the left sensing glove. FIG. 3 is an example of a cross section of the sensing device 100. While a sensing glove is shown and described by way of example, in other cases the sensing device 100 may be another type of sensing device, such as a sleeve, hat, glasses, shirt, wristband, watch, ring, belt, sock, shoe, etc.

The sensing device 100 may be a multimodal sensing device capable of simultaneously sensing multiple types of data from multiple types of sensors at a same sampling time, such as force, motion, temperature, proximity, imaging, and/or conductivity sensing. The sensing device 100 may comprise a wearable article, such as a glove including a plurality of digits 102A-102E (e.g., fingers corresponding to digits 102A-102D, and a thumb corresponding to digit 102E), a hand portion 106, and a wrist portion 108, coupled to one another, with an opening to receive a hand of a user. Each of the digits 102A-102E may include a plurality of digit sections defined relative to moveable joints, such as a digit section 110A between a metacarpophalangeal (MCP) joint and a proximal interphalangeal (PIP) joint, digit section 110B between the PIP joint and a distal interphalangeal (DIP) joint, and digit section 110C forward of the DIP joint (e.g., the fingertip). The digit 102E (thumb) may also include digit sections relative to joints, such as digit section 112A between an MCP joint and an interphalangeal (IP) joint, and digit section 112B forward of the IP joint (e.g., the thumb tip).

As illustrated in FIG. 1 (palmar side of left sensing glove), the sensing device 100 includes a plurality of sensor arrays 114. Each sensor array may include a plurality of sensors 115 (e.g., microsensors). Each sensor array 114 may utilize one or more of the sensors 115 to obtain a biometric measurement, such as force data indicating a force or pressure applied to the sensing device 100 in a sensing area. In some cases, a sensor array 114P may also be coupled to a palmar side of the hand portion 106 of the sensing glove (e.g., another section, such as the palm). The sensor arrays 114 may enable tactile sensing via sensors 115, such as normal force sensors and/or shear force sensors, to replicate human-scale tactile sensing, touch, grasp, and/or dexterity. Each sensor array 114 may be coupled to a wearable article 101 of the sensing device 100 (e.g., at a digit section 110 or the palm). For example, the wearable article 101 could be a fabric, elastomer, or other textile.

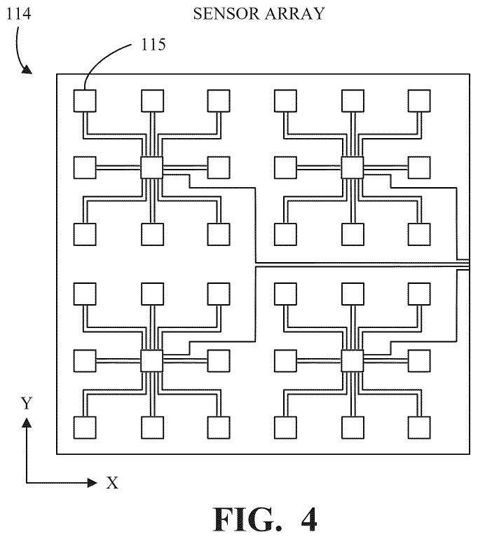

With additional reference to FIG. 4, the sensors 115 in a sensor array 114 may be submillimeter in at least one in-plane dimension (e.g., the X and Y axes shown) associated with a footprint of the sensor on the sensing device 100. Further, the sensors 115 may have a pitch (e.g., distance between sensors, or from one footprint to another) of 3 mm or less, and in some cases, a pitch of 1 mm or less, to enable a high density of sensing. Additionally, the sensors 115 may be distributed at multiple locations of the sensing area of the wearable article 101, such as a grid of rows and columns of sensors in a digit section, palm, etc. The sensors are shown in a 6×6 grid by way of example. The multiple locations may be given by coordinates, such as coordinates A11 to A66 corresponding to sensors 115 in a grid of rows and columns (e.g., positions along X and Y axes). For example, a first row, first sensor may have a coordinate A11, a first row, second sensor may have a coordinate A12, and so forth. In some implementations, the sensing device 100 may include more than 1,000 of the sensors 115 on the outer surface, and in some cases, more than 10,000 of the sensors 115.

In some implementations, the sensors 115 may be configured as force sensors, temperature sensors, proximity sensors, image sensors, and/or conductivity sensors (e.g., multimodal sensing). In some examples, a sensor 115 could be a piezoelectric sensor, piezoresistive sensor, metal foil strain sensor (e.g., strain gauge), capacitive sensor, etc. to obtain force data indicating a force applied to the sensor. This may enable tactile sensing. Further, the sensors 115 may enable a high dynamic sensing range, such as a pressure range that encompasses a skin puncture threshold and a lower limit of human touch. For example, the pressure range includes 1.5 mg/mm² to 100 g/mm². Additionally, the sensing device 100 may be substantially thin, e.g., less than 2 mm, and flexible for unobtrusive sensing while worn by the user. In other examples, a sensor 115 could be a temperature sensor (e.g., a piezoelectric sensor utilizing the pyroelectric effect), a proximity sensor (e.g., a capacitive sensor responsive to proximity of an object), an image sensor (e.g., a photo sensitive elements), and/or a conductivity sensor (e.g., an exposed electrode), and in some cases, may include or be replaced with an LED at a location.

Each sensor can individually generate a digital output indicating data from a sensing element, such as force data from force sensors, temperature data from temperature sensors, proximity data from proximity sensors, image data from image sensors, and/or conductivity data from conductivity sensors. The digital outputs of the sensors 115 may be read out, along with sensors 115 from other sensor arrays 114, by readout circuitry coupled to a processor 120. The data from the sensors 115 may enable biometric measurements to be obtained based on a spatial map of sensed conditions from the sensors 115 at the multiple locations. This may include force, pressure, temperature, proximity, image, and/or conductivity distributions.

In some cases, the sensors 115 may be connected in series (e.g., a daisy chain), and in some cases, the sensors 115 may be connected in parallel (e.g., rows and columns). In some cases, the sensors 115 may include LEDs or may be replaced with LEDs (e.g., to emit light to the skin). In some cases, the sensors 115 may include exposed electrodes (e.g., to sense conductivity of the skin). In some cases, a cluster of sensors 115 of a sensor array 114 may be utilized together to form a single point of sensing. For example, two or more sensors 115 of a sensor array 114 may act together to sense a force. In another example, multiple sensors 115 (and/or LEDs) of a sensor array 114 may act together to produce light and/or sense light, such as to sense a blood flow.

Some sensor arrays 114 may have a higher density of sensors 115 per unit area than other sensor arrays 114. For example, sensor arrays 114, coupled to digit sections 110A-110E, may have a higher density of sensors per unit area, such as a 1×1 mm pitch between sensors 115, than sensor array 114P coupled to the palm, which may have a 5×5 mm pitch between sensors 115. Conversely, some sensor arrays 114 may have a lower density of sensors 115 per unit area than other sensor arrays 114, such as the sensor array 114P.

Referring again to FIG. 2 (dorsal side of left sensing glove), the sensing device 100 may include a plurality of motion sensors 116. For example, each motion sensor 116 could be a multi-axis IMU, such as a nine-axis IMU, that senses one or more motions of the sensing device 100. Each motion sensor 116 may be coupled to the wearable article 101 at a digit or digit section, arranged on dorsal sides of each finger. Further, one or more additional motion sensors 116 may be arranged on the dorsal side of the wrist portion 108, such as motion sensor 116W.

Each motion sensor 116 can individually generate its own digital output indicating motion data that may be read out by digital readout circuitry, such as the processor 120. The motion sensors 116 may enable measurements including a position, orientation, trajectory, velocity, and/or acceleration of each digit section relative to the wrist portion 108, and/or an absolute position, orientation, trajectory, velocity, and/or acceleration. The sensing device 100 may also include a microphone 136 and/or a button 137 for environmental sensing and/or receiving user input.

In some implementations, the processor 120 can execute to obtain a gait measurement of a user from one or more motion sensors 116. For example, motion sensor 116W coupled to the wrist portion 108 of the wearable article 101 may enable the processor 120 to obtain a gait measurement as the user is walking or running. The processor 120 can then sense a gait change, such as to detect an injury to foot or leg. In some implementations, the processor 120 can obtain digit measurements relative to one another from one or more motion sensors 116. For example, motion sensors 116 coupled to digit sections of digits may enable the processor 120 to obtain finger motions, such as an evaluation of typing, or evaluation of a health condition, such as Parkinson's disease or dementia.

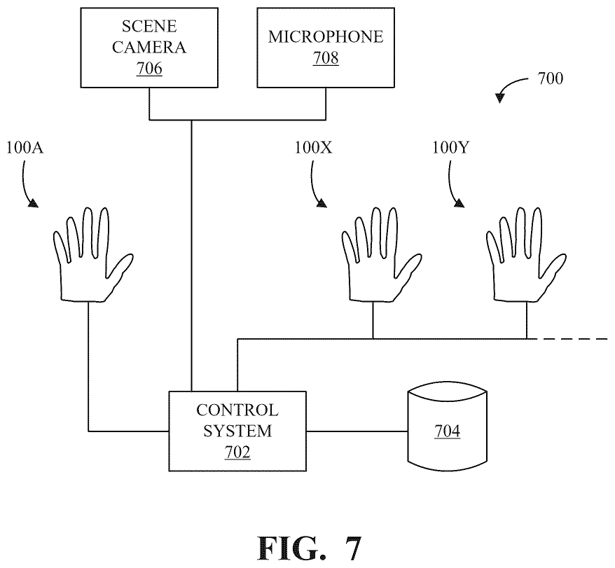

In some implementations, the sensing device 100 may utilize a plurality of optical markers 118 for tracking positions of the digit sections. For example, each optical marker 118 may be coupled to a digit section, arranged on a dorsal side of a finger or thumb. Further, one or more optical markers 118 may be arranged on the dorsal side of the wrist portion 108, such as optical sensor 118W. As shown, the optical markers 118 may couple with the motion sensor 116, centered on the finger and thumb sections and the wrist portion 108 and the motion sensor 116 location. The optical markers 118 may enable a system utilizing a scene camera (e.g., scene camera 706 in FIG. 7) or other form of detection to determine a position of each digit section 110 relative to the wrist portion 108.

The sensing device 100 may also include circuitry, such as the processor 120 and memory, a communications device 122, and a power source 124. For example, the processor 120 and memory may be implemented by a system on a chip (SoC), application specific integrated circuit (ASIC), or other integrated circuit (IC) coupled to the sensing device 100. The communications device 122 may be a wireless communications device implemented by an IC coupled to the sensing device 100. In some implementations, the communications device 122 may enable an IEEE 802.X communications protocol (e.g., Wi-Fi, Bluetooth, or ZigBee). The power source 124 may include a battery, power supply, charging circuitry, and/or a port for wired and/or wireless charging. The power source 124 may power the circuitry of the sensing device 100, including the processor 120, memory, and communications device 122, and the sensors. The power source 124 and the communications device 122 may enable convenient, fully wireless operation of the sensing device 100 by a user.

Referring again to FIG. 3, in various configurations, sensors 115 of the sensor arrays 114 may be outward facing from the wearable article 101 (coupled to an outer surface), such as sensor array 114A; inward facing within the wearable article 101 (coupled to an inner surface), such as sensor array 114B; or a combination of both. For example, in an outward facing configuration, sensors 115 of sensor array 114A may be coupled to a flexible circuit 132A that is coupled to the sensing device 100. This configuration may enable the sensor array 114A to contact a surrounding environment 170, such as to perform as a sensing/diagnostic tool for the user to utilize with another individual. The flexible circuit 132A may have strain reliefs (e.g., cutouts) between sensors 115 to enable flex and bending of the mounted circuitry to form a deformable sensor array. The flexible circuit 132A may couple with an exterior surface of the wearable article 101 via an adhesive 131A. Further, the flexible circuit 132A and the sensor array 114A may be sealed via a flexible encapsulation 133A such as silicone. Wiring between the sensors 115 and the readout circuitry, such as electrodes 134, may wrap around the exterior surface of the wearable article 101, from the front side (palmar) to the back side (dorsal), in a serpentine or zig zag pattern. The electrodes 134 may enable connections around joints, between each sensor 115 of each sensor array 114A to digital readout circuitry, while enabling the flexing, bending, and conformance of the sensing device 100.

In another example, in an inward facing configuration, sensors 115 of sensor array 114B may be coupled to a flexible circuit 132B coupled to the sensing device 100. This inward facing configuration may enable the sensor array 114B to directly contact the user 128 wearing the wearable article. This may enable the sensing device 100 to perform as a sensing/diagnostic tool for the user that is wearing the device. The flexible circuit 132B may have strain reliefs (e.g., cutouts) between sensors 115 to enable flex and bending of the mounted circuitry to form another deformable sensor array. The flexible circuit 132B may couple with an interior surface of the wearable article 101 via an adhesive 131B. Further, the flexible circuit 132B and the sensor array 114B may be sealed via a flexible encapsulation 133B such as silicone. Wiring between the sensors 115 and the readout circuitry, such as electrodes 134, may wrap around the interior surface of the wearable article 101, from the front side (palmar) to the back side (dorsal), in a serpentine or zig zag pattern. The electrodes 134 may enable connections around joints, between each sensor 115 of each sensor array 114B to digital readout circuitry, while enabling the flexing, bending, and conformance of the sensing device 100.

In some implementations, a combination of inward facing and outward facing sensor arrays 114 may enable biometric measurements of one sensor array to compensate for another (e.g., an isolation of readings from sensors). For example, the sensing device 100 may be utilized to obtain biometric measurements of another individual in the surrounding environment 170 via the sensor array 114A (outward facing). Such biometric measurements may be compensated for based on the user's own biometric measurements obtained via the sensor array 114B (inward facing). In another example, the sensing device 100 may be utilized to obtain biometric measurements of the user 128 via the sensor array 114B (inward facing). In this case, such biometric measurements may be compensated for based on environmental conditions in the surrounding environment 170 obtained via the sensor array 114A (outward facing). In some cases, a thermal insulation layer 141 may be arranged between inward facing sensor arrays (e.g., the sensor array 114B) and outward facing sensor arrays (e.g., the sensor array 114A) to provide high thermal resistance between the sensor arrays.

Additionally, each motion sensor 116 may be coupled to a flexible circuit 132C coupled to the sensing device 100. The flexible circuit 132C may also have strain reliefs (e.g., cutouts) between motion sensors 116 to enable flex and bending of the mounted circuitry. The flexible circuit 132C may couple with the wearable article 101 via an adhesive 131C. Further, the flexible circuit 132C, and the motion sensors 116, may be sealed via a flexible encapsulation 133C, such as silicone. Wiring between the motion sensors 116 and the readout circuitry, such as electrodes 134, may wrap around joints of the sensing device 100 in a serpentine or zig zag pattern. The electrodes 134 may enable connections between each motion sensor 116 to digital readout circuitry, while enabling flexing, bending, and conformance of the sensing device 100.

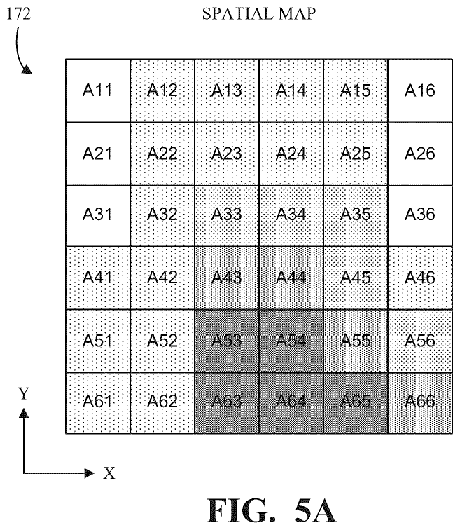

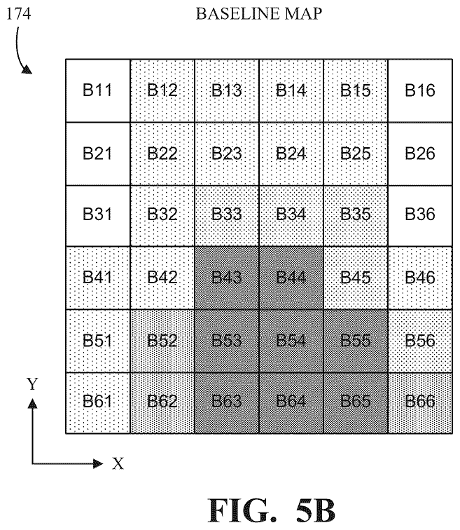

In operation, the processor 120 can execute instructions stored in memory to obtain, evaluate, and/or monitor biometric measurements via the sensors of the sensing device 100. For example, with additional reference to FIG. 5A, a biometric measurement may include a spatial map 172 of sensed conditions from sensors 115 at multiple locations of a sensor array 114 (e.g., a heat map). The sensed conditions are shown in a 6×6 grid with each sensed condition corresponding to a sensor 115 in the 6×6 grid of sensors shown in FIG. 4. The sensed conditions may have a resolution corresponding to the pitch of the sensors 115, such as each sensed condition spanning 3 mm or less, and in some cases, 1 mm or less. The sensors 115 being submillimeter in at least one in-plane dimension (e.g., the X and Y axes shown) may enable localized, high resolution sensing. The multiple locations may be given by coordinates, such as coordinates A11 to A66 corresponding to sensors 115 in a grid of rows and columns (e.g., positions along X and Y axes, each indicating a biometric measurement value corresponding to a sensed condition from a sensor). The processor 120 can utilize the readout circuitry to obtain the biometric measurement from the sensors and sensor arrays and determine the spatial map 172 (e.g., reading the digital outputs the sensors 115 and/or motion sensors 116 at a sampling time). Further, the processor 120 can assign a timestamp to the digital outputs corresponding to the biometric measurement. A sensed condition indicated by a digital output of a sensor 115 may be classified and/or quantified in a respective area of the spatial map 172. For example, sensors 115 at coordinates A53, A54, A63, 664, and A65 might be quantified as maximum values (indicating saturation), whereas sensor 115 at coordinates A11, A21, A31, A16, A26, and A36 might be quantified as minimum values (e.g., indicating no detection), with other sensors 115 quantified with values in between.

With additional reference to FIG. 5B, the processor 120 can further execute to generate an output (feedback) based on comparing the spatial map 172 of sensed conditions to a baseline map 174 of reference values at corresponding locations. The reference values may have a resolution corresponding to the pitch of the sensors 115, such as each reference value spanning 3 mm or less, and in some cases, 1 mm or less. The multiple locations may be given by coordinates, such as coordinates B11 to B66 (e.g., positions along X and Y axes), each indicating reference value corresponding to one of the coordinates A11 to A66 of the spatial map 172. In the comparison, the processor 120 can calculate deviations to determine whether sensed conditions match corresponding reference values to within a threshold (a match) or differ from the corresponding reference values by more than the threshold (a mismatch). In some cases, the threshold may be configured based on the sensing application, such as sensing blood oxygen level, heart rate, blood pressure, blood flow, skin/body temperature, skin conductivity, confirming identity, enabling equipment, linking performance to users, etc. In some cases, the threshold can be controlled based on user input (e.g., adjusting sensitivity), and in some cases, can be controlled by a prediction generated by a machine learning model (e.g., predicted based on the user 128 that is wearing the sensing device 100 and/or the sensing application that is selected).

For example, coordinate A11 of the spatial map 172 (biometric measurement value) and coordinate B11 of the baseline map 174 (reference value), corresponding to one another, may both indicate minimum values resulting in an exact match. Also, coordinate A63 of the spatial map 172 and coordinate B63 of the baseline map 174, corresponding to one another, may both indicate maximum values resulting in an exact match. However, coordinate A55 of the spatial map 172 may indicate some intermediate value, whereas corresponding coordinate B55 of the baseline map 174 may indicate another maximum value. An output may then indicate a match or a mismatch (corresponding to this sampling time) based on the threshold configured for the application. For example, in some cases, an intermediate biometric measurement value at a coordinate location may be determined to be a mismatch when compared to a maximum reference value, whereas in other cases the intermediate biometric measurement may be close enough to the maximum reference value to determine a match.

In some cases, sensed conditions differing from reference values by more than a threshold can cause an output that includes sending an alert. For example, when the biometric measurement indicates a blood oxygen level, heart rate, blood pressure, blood flow, skin/body temperature, or skin conductivity that exceeds a threshold (e.g., a predetermined target), the output can include an alert that indicates exceeding the target (e.g., a warning to the user 128, a health provider (e.g., doctor), a supervisor, etc., of deviations which may indicate fatigue, over exertion, or a health issue). Conversely, the reference values matching from the sensed conditions to within the threshold can cause an output that simply indicates maintaining the target at the sampling time.

In some cases, the sensed conditions matching or differing from the reference values can cause an output that confirms or rejects an identity of a user, respectively. For example, the reference values may indicate a blood oxygen level, heart rate, blood pressure, blood flow, body temperature, or skin conductivity that may be unique to a user, analogous to fingerprints. The sensed conditions matching the reference values to within a threshold can confirm the identity of the user as matching the user that provided the reference values. Conversely, the sensed conditions differing from the reference values by more than a threshold can indicate the identity of the user does not match the user that provided the reference values.

In some cases, the sensed conditions matching or differing from the reference values can cause an output that enables or disables equipment (e.g., the sensing device 100) and/or connects or disconnects performance of the equipment to the user. For example, the reference values may indicate a blood oxygen level, heart rate, blood pressure, blood flow, body temperature, or skin conductivity that may be unique to a user, analogous to fingerprints. The sensed conditions matching the reference values to within a threshold can enable the equipment to be utilized (e.g., activated or turned on) and/or performance of the equipment to be connected or linked to a user (e.g., a profile of the user). This can enable secure use and efficient tracking of performance by the user when using the sensing device 100 to perform a task, such as manufacturing a wearable article. Conversely, the sensed conditions differing from the reference values by more than a threshold can disable the equipment and/or disconnect the equipment from being linked to the user (and instead enabling equipment and/or linking to another user).

In some cases, the processor 120 may execute to generate an alert based on the output that includes illuminating a multi-color light emitting diode (LED) 135 and/or applying haptic feedback via haptic actuator 139. The alert can signal to the user, via the sensing device 100, that the sensed conditions differ from the reference values by more than a threshold.

In some implementations, the processor 120 can balance between power consumption and sensing resolution of the sensing device 100. For example, the processor 120 can select between different scanning modes at separate times, such as a coarse scan or a fine scan. The coarse scan can enable fewer sensors 115 of a sensor array 114 to be read out to conserve power from the power source 124 (e.g., utilizing alternating sensors 115, so that every other sensor 115 may be in a low power mode). The fine scan can enable more sensors of the sensor array to be read out to increase sensing resolution of the spatial map 172 (e.g., utilizing all sensors 115, so that a maximum resolution may be achieved). The processor 120 can balance power consumption and sensing resolution for a given application by determining which sensors and/or sensor arrays to utilize. Further, the processor 120 can balance between power consumption and sampling rates by selectively performing scans at a predetermined sampling frequency, such as one scan per hour, minute, or second.

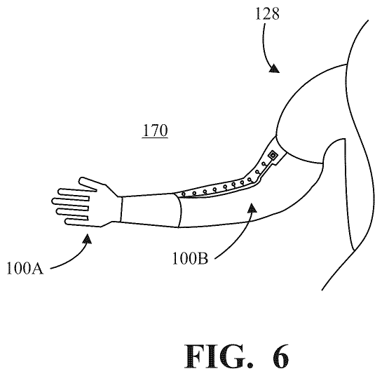

FIG. 6 is an example of the user 128 wearing multiple sensing devices 100, such as a sensing device 100A and a sensing device 100B. For example, the sensing device 100A may be a sensing glove like the sensing glove shown in FIGS. 1 and 2, worn on a hand of the user 128. The sensing device 100B may be another sensing device, such as a sleeve worn on an arm of the user 128 (e.g., a compression garment). In other cases, a sensing device may include a hat, glasses, shirt, wristband, watch, ring, belt, sock, shoe, etc. The sensor arrays 114 of the sensing devices 100, coupled to the flexible substrates described in FIG. 3, can stretch, bend, and fold to follow contours of the user 128 and may be deformable with motion of the user's body. As a result, the sensor arrays 114 can sense conditions from the sensors 115 at multiple locations of the user corresponding to the sensing areas, and obtain biometric measurements from the user, pursuant to a variety of motions, activities, and tasks of the user.

In some cases, the processor 120 can compress and encode a biometric measurement in a bitstream, and/or transmit (via the communications device 122) the biometric measurement to a system to obtain an output (feedback) from the system. For example, the processor 120, via the communications device 122, can relay obtained biometric measurements to a control system which may include a remotely located storage device. The processor 120 can relay the measurements with timestamps, e.g., at a rate of up to 100 Hz, while the user wears the sensing device 100 to perform a task. The processor 120, via the communications device 122, can then receive the output from the control system corresponding to the timestamps.

By way of example, FIG. 7 is a system 700 in which one or more sensing devices 100 may operate. The system 700 may include a control system 702, a storage device 704, a scene camera 706, a scene microphone 708, and a plurality of sensing devices, such as sensing devices 100A, 100X, 100Y, and so forth, shown by way of example. The storage device 704 can store baseline maps of reference values corresponding to users and/or user profiles. The control system 702 can utilize biometric measurements from one or more the sensing devices to generate outputs, individually or in connection with one another. The control system 702 can also utilize the environmental sensors to generate the output, such as the scene camera 706 and/or the scene microphone 708. For example, to confirm or reject an identity of a user, and/or to link performance of equipment to a profile, the control system 702 can utilize biometric measurements from a sensing device, along with facial recognition from the scene camera 706, and/or voice patterns from the scene microphone 708, in concert with one another. In another example, to assess a physiological condition of a user, the control system 702 can utilize biometric measurements from a sensing device to obtain blood oxygen level, heart rate, blood pressure, blood flow, skin/body temperature, or skin conductivity, and utilize the scene camera 706 and/or the scene microphone 708 to determine activity of the user, e.g., walking, exercising, sleeping, working, etc.

In some cases, the output (feedback) for one sensing device may be generated by the control system 702 based on comparing biometric measurements to another sensing device, e.g., a primary sensing device. For example, sensing device 100A may be a primary sensing device worn by a primary user, sensing device 100X may be a secondary sensing device worn by another user, sensing device 100Y may be another secondary sensing device worn by yet another user, and so forth. The biometric measurements (e.g., the spatial map 172) from sensing device 100A (primary) may be used to generate the baseline maps (e.g., the baseline map 174) to which biometric measurements from secondary sensing device may be compared (e.g., sensing devices 100X, 100Y, etc.). This may enable a primary user to set a standard for other users, such as performing a task with an object in a particular way. For example, when a secondary user performs the task with the object in a different way (e.g., exerting too much force, traveling with too much motion, etc.), the control system 702 can determine the difference and generate an output that includes an alert to the secondary user.

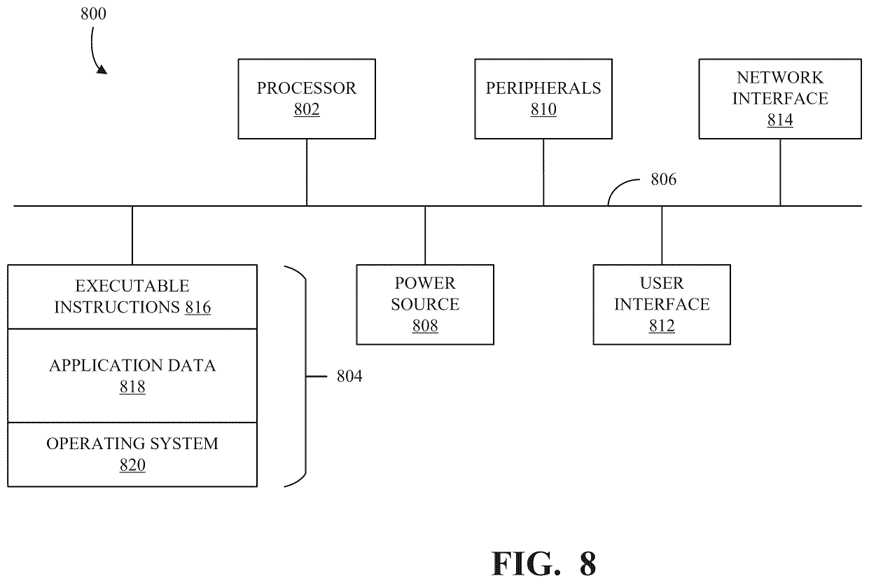

FIG. 8 is a block diagram of an example internal configuration of a computing device 800 for utilizing a sensing device 100. For example, the computing device 800 could be implemented by the sensing device 100 and/or the control system 702. The computing device 800 includes components or units, such as a processor 802, a memory 804, a bus 806, a power source 808, peripherals 810, a user interface 812, a network interface 814, other suitable components, or a combination thereof. One or more of the memory 804, the power source 808, the peripherals 810, the user interface 812, or the network interface 814 can communicate with the processor 802 via the bus 806.

The processor 802 is a central processing unit, such as a microprocessor, and can include single or multiple processors having single or multiple processing cores. Alternatively, the processor 802 can include another type of device, or multiple devices, configured for manipulating or processing information. For example, the processor 802 can include multiple processors interconnected in one or more manners, including hardwired or networked. The operations of the processor 802 can be distributed across multiple devices or units that can be coupled directly or across a local area or other suitable type of network. The processor 802 can include a cache, or cache memory, for local storage of operating data or instructions.

The memory 804 includes one or more memory components, which may each be volatile memory or non-volatile memory. For example, the volatile memory can be random access memory (RAM) (e.g., a DRAM module, such as dual data rate (DDR) DRAM). In another example, the non-volatile memory of the memory 804 can be a disk drive, a solid state drive, flash memory, or phase-change memory. In some implementations, the memory 804 can be distributed across multiple devices. For example, the memory 804 can include network-based memory or memory in multiple clients or servers performing the operations of those multiple devices.

The memory 804 can include data for immediate access by the processor 802. For example, the memory 804 can include executable instructions 816, application data 818, and an operating system 820. The executable instructions 816 can include one or more application programs, which can be loaded or copied, in whole or in part, from non-volatile memory to volatile memory to be executed by the processor 802. For example, the executable instructions 816 can include instructions for performing some or all of the techniques of this disclosure. The application data 818 can include user data, database data (e.g., database catalogs or dictionaries), or the like. In some implementations, the application data 818 can include functional programs, such as a web browser, a web server, a database server, another program, or a combination thereof. The operating system 820 can be, for example, any known personal or enterprise operating system; an operating system for a mobile device, such as a smartphone or tablet device; or an operating system for a non-mobile device, such as a mainframe computer.

The power source 808 provides power to the computing device 800. For example, the power source 808 can be an interface to an external power distribution system. In another example, the power source 808 can be a battery, such as where the computing device 800 is a mobile device or is otherwise configured to operate independently of an external power distribution system. In some implementations, the computing device 800 may include or otherwise use multiple power sources. In some such implementations, the power source 808 can be a backup battery.

The peripherals 810 includes one or more sensors, detectors, or other devices configured for monitoring the computing device 800 or the environment around the computing device 800. For example, the peripherals 810 can include a geolocation component, such as a global positioning system location unit. In another example, the peripherals can include a temperature sensor for measuring temperatures of components of the computing device 800, such as the processor 802. In some implementations, the computing device 800 can omit the peripherals 810.

The user interface 812 includes one or more input interfaces and/or output interfaces. An input interface may, for example, be a positional input device, such as a mouse, touchpad, touchscreen, or the like; a keyboard; or another suitable human or machine interface device. An output interface may, for example, be a display, such as a liquid crystal display, a cathode-ray tube, a light emitting diode display, virtual reality display, or other suitable display.

The network interface 814 provides a connection or link to a network. The network interface 814 can be a wired network interface or a wireless network interface. The computing device 800 can communicate with other devices via the network interface 814 using one or more network protocols, such as using Ethernet, transmission control protocol (TCP), internet protocol (IP), power line communication, an IEEE 802.X protocol (e.g., Wi-Fi, Bluetooth, or ZigBee), infrared, visible light, general packet radio service (GPRS), global system for mobile communications (GSM), code-division multiple access (CDMA), Z-Wave, another protocol, or a combination thereof.

Reference is now made to a flowchart of an example of a process for utilizing sensing system to sense, evaluate, and/or monitor biometric measurements. The process can be executed using computing devices, such as the systems, hardware, and software described with respect to FIGS. 1-8. The process can be performed, for example, by executing a machine-readable program or other computer-executable instructions, such as routines, instructions, programs, or other code. The operations of the process or other techniques, methods, or algorithms described in connection with the implementations disclosed herein can be implemented directly in hardware, firmware, software executed by hardware, circuitry, or a combination thereof.

For simplicity of explanation, the process is depicted and described herein as a series of operations. However, the operations in accordance with this disclosure can occur in various orders and/or concurrently. Additionally, other operations not presented and described herein may be used. Furthermore, not all illustrated operations may be required to implement a process in accordance with the disclosed subject matter.

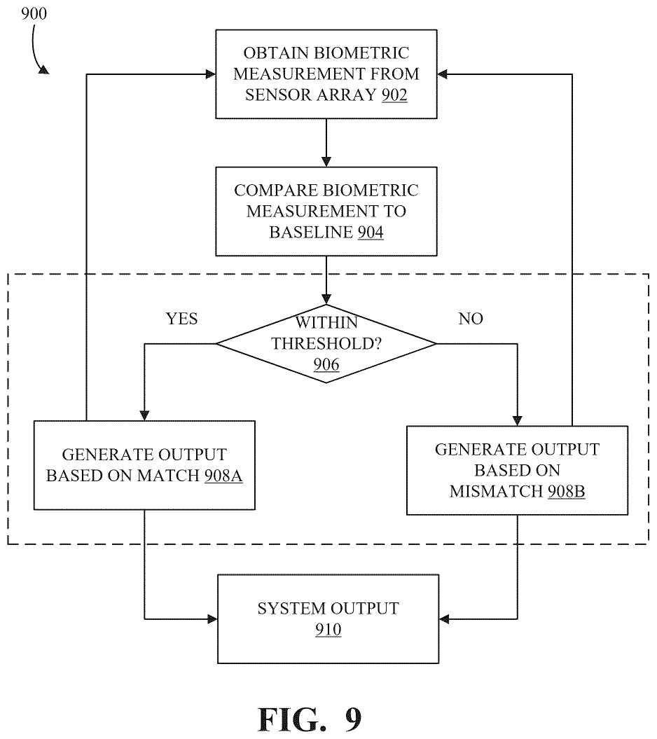

FIG. 9 is an example of a process 900 for utilizing a sensing device. At operation 902, a processor can obtain a biometric measurement from a sensing device 100. The processor could be implemented by the sensing device 100 or by another control system, such as a mobile device, server, or cloud based computing system (e.g., the control system 702). The sensing device 100 may include a sensor array 114 coupled to a wearable article (e.g., a glove, sleeve, hat, glasses, shirt, wristband, watch, ring, belt, sock, shoe, etc.). The sensor array 114 may include sensors 115 that are submillimeter in at least one in-plane dimension (e.g., microsensors) and are distributed at multiple locations of a sensing area of the wearable article (e.g., a fingertip, thumb tip, or palm of a glove, or patch of a sleeve, garment, or other wearable article). The biometric measurement may include a spatial map 172 of sensed conditions from the sensors 115 at the multiple locations of the sensing area. In some cases, the biometric measurement may be of a plurality of biometric measurements obtained over an extended period of time, such as periodically during a multi-hour shift (e.g., 8 hours), multiple days of a week, or multiple weeks of a month.

At operation 904, the processor can compare the spatial map 172 to a baseline map 174 of reference values corresponding to the multiple locations. This may include determining deviations between sensed conditions and reference values at corresponding locations.

At operation 906, the processor can determine whether the sensed conditions match the reference values to within a threshold. If the sensed conditions match the reference values to within a threshold (Yes), at operation 908A the processor can generate a first output (positive feedback) based on the match. This can result in a first type of system output at operation 910. For example, this may include an indication of maintaining a blood oxygen level, heart rate, blood pressure, blood flow, skin/body temperature, or skin conductivity relative to a predetermined target. In some cases, this may include confirming an identity of a user. In some cases, this may include enabling equipment (e.g., the sensing device 100) and/or enabling performance of equipment (e.g., the sensing device) to be linked to the user (e.g., a profile of the user).

However, if at operation 906 the sensed conditions do match the reference values to within a threshold (No), and instead differ from the reference values by more than a threshold, at operation 908B the processor can generate a second output (negative feedback) based on the mismatch. This can result in a second type of system output at operation 910. In some cases, this may include an indication of the blood oxygen level, heart rate, blood pressure, or body temperature exceeding the predetermined target. In some cases, this may include focusing an intervention, medication, etc. to a sensing area associated with the user or other individual. In some cases, this may include rejecting an identity of a user. In some cases, this may include disabling equipment (e.g., the sensing device 100) and/or disabling performance of equipment (e.g., the sensing device) from being linked to the user (e.g., the profile of the user). In some cases, this may include sending an alert indicating the sensed conditions differing from the reference values by more than a threshold. For example, the alert may include illumination of an LED, haptic feedback, transmission of a message via a network, etc.

In some cases, the sensing device 100 may be used by a manufacturer, supervisor, or trainer as a training tool in a manufacturing environment or teaching environment. For example, biometric measurements from sensor arrays 114 and/or motions sensors 116 may enable the processor 120 to detect changes in applied forces or motions of the user over time (relative to baseline maps) to indicate in an output a change in productivity (e.g., due to fatigue).

In some cases, the sensing device 100 may be used by a medical practitioner as a diagnostic tool in a clinical setting. For example, during a routine physical examination, a medical practitioner could scan the sensing device 100 over a patient to enable the processor 120 to determine anomalies in spatial maps of biometric measurements of the patient (relative to baseline maps, which may be idealized). In another example, if a patient indicates a specific issue, such as a loss of touch, the sensing device 100 can provide high-spatial resolution quantitative data to assist a diagnosis, such as by determining that the patient cannot feel presses less than 100 gram-force, indicating possibilities of peripheral neuropathy and/or diabetes. In another example, the processor 120 can analyze time series data collected from a patient wearing the sensing device 100 over extended periods of time, e.g., periodically during a multi-hour shift, multiple days of a week, or multiple weeks of a month, to determine deviations and/or trends in the measurements.

In some cases, the sensing device 100 may be used to indicate a condition of the user or another individual. For example, biometric measurements from sensor arrays 114 coupled to digit sections of digits may enable the processor 120 to obtain forces at predetermined sampling times to detect forces at fingertips of the user (relative to baseline maps, such as 100 gram-force) which may indicate a possible condition of peripheral neuropathy or diabetes. In another example, biometric measurements from sensor arrays 114 may enable the processor 120 to obtain forces at predetermined sampling times to detect anomalies in blood pressure of the user or another individual being tested (relative to baseline maps), which may indicate a possible condition of a blood clot. In another example, biometric measurements from motion sensors 116 coupled to digit sections of digits may enable the processor 120 to obtain hand and finger motions at predetermined sampling times to detect finger tremors, an unstable gait of the user, etc., (relative to baseline maps), which may indicate a possible condition of Parkinson's disease or dementia.

In some cases, a combination of wide area ultrasonic imaging, temperature, and pressure, the glove could give insight into subdermal bodily tissue irregularities/anomalies. For example, biometric measurements from sensor arrays 114 dispersed over a wide area may enable the processor 120 to obtain a combination of sensing at predetermined sampling times, such as temperatures and pressures. This may enable detecting local areas of inflamed tissue of the user or another individual being tested (relative to baseline maps), which may indicate a possible condition of a tumor, cancerous tissue, or other local anomaly. For example, a tumor, cancerous tissue, or other local anomaly may present both firmer tissue relative to surrounding tissue and a temperature difference of the tissue relative to the surrounding tissue. This signature can be detected in the biometric measurements, and then compared to a baseline map, to determine presence or absence of a possible condition.

In some cases, the sensor arrays 114 of the sensing device 100 may operate synchronously with one another to form a large-area sensing device, such as an ultrasound imager. This may enable diagnostics, such as mammograms, skin exams, etc. For example, sensors of the sensor arrays 114 may be time multiplexed to perform in different modes at different times, such as piezoelectric sensors operated in an actuator mode to generate acoustic energy (ultrasonic waves) during a first time, then operated in a sensing mode to sense the acoustic energy during a second time. Further, sensors of the sensor arrays 114 may be operated together or ganged to perform the different functions at the different times. For example, multiple piezoelectric sensors may be ganged together during the first time in the actuation mode to produce an increased amount of acoustic energy to be sensed, then ganged together during the second time in the sensing mode to produce an increased amount of sensing area to sense the acoustic energy.

In some cases, the sensing device 100 may long term monitoring of a user or another individual (e.g., multiple days of a week, or multiple weeks of a month, etc.). For example, a user (patient) could self-perform periodic palpitations while wearing the sensing device 100 in a private setting. The system may generate a baseline map of initial conditions sensed by the sensing array, such as an initial tissue condition using temperature and/or force sensing at t=0. As the user self-checks over time (e.g., weeks, months, etc.), the system can compare subsequent temperature and/or force sensing over time and notify the user if an anomaly is detected. This may enable the sensing device 100 to be used as a preventative early detection tool to inform a patient and/or medical practitioner of a change in between office visits.

In some cases, the biometric measurements may be utilized to assist in the performance of tasks with objects in a work environment. For example, a primary user wearing a primary sensing device (e.g., sensing device 100A) can obtain a series of biometric measurements to establish baseline maps of reference values. A secondary user wearing a secondary sensing device (e.g., sensing device 100X) can obtain a series of biometric measurements to obtain spatial maps of sensed conditions. A control system can generate an output that includes feedback to the secondary sensing device (and the secondary user) to change performance with the secondary sensing device to more closely match the performance of the primary sensing device.

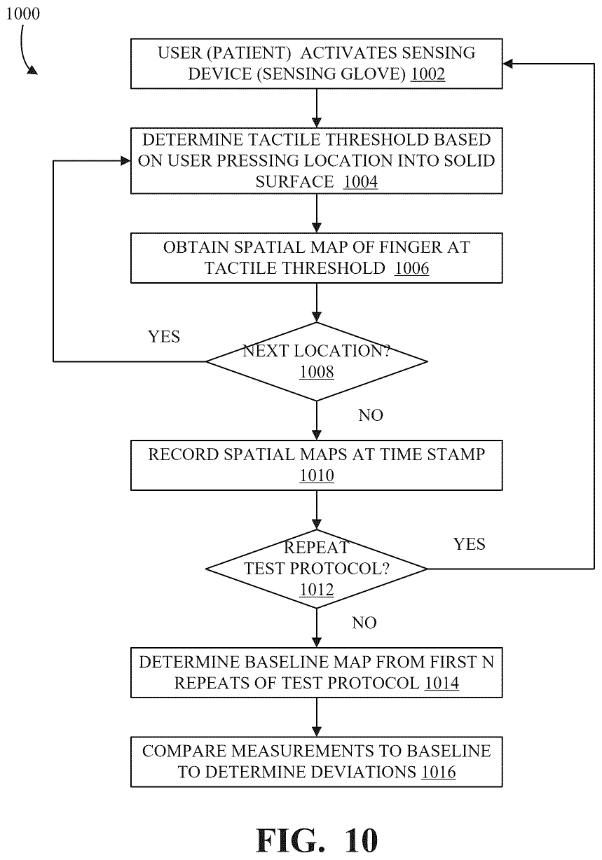

FIG. 10 is an example of a first process 1000 (a test protocol, or test) for determining peripheral neuropathy of an individual, utilizing the sensing device 100. At operation 1002, a user (e.g., a patient) activates the sensing device 100 (e.g., a sensing glove), such as by donning the sensing device 100. For example, the sensing device 100 may include the sensor array 114B inward facing to detect forces at an interior surface of the sensing device. At operation 1004, the system may determine a tactile threshold for a location (e.g., a finger, fingertip, thumb, thumb tip, toe, etc.) of the user based on the user pressing the location into a solid surface, such as a table, gradually increasing applied force until the user can feel the surface and provide feedback (e.g., detected by the system as a cue from the user, such as via the microphone 136). At operation 1006, the system may obtain a biometric measurement to produce/record a spatial map of the location that corresponds to the tactile threshold (e.g. a force or pressure map). At operation 1008, the system may determine whether to repeat the measurement for a next location (e.g., repeat if less than five digits measured on the hand, less than 10 digits measured in total, etc.). If the system determines to repeat the measurements (Yes), the system may return to operation 1004 for the next location. However, if the system determines not to repeat the measurement (No), at operation 1010 the system may record spatial maps for each of the location (e.g., five digits per hand, 10 digits total, etc.) corresponding to a time stamp, such as a particular date and time. At operation 1012, the user may repeat the test periodically, one or more times over the course of weeks, months, or years. In some cases, the system may generate an alert to the user as a reminder to repeat the test, which may be configured at the direction of a health provider. If the user repeats the test (Yes), the test may be performed again beginning at operation 1002. However, if the user does not repeat the test (No), at operation 1014 the system may determine a baseline measurement (e.g., a baseline map of reference values) for the user, from the first N repeats of the test, where N is an integer greater than or equal to one. In some cases, N may be determined by the health provider's instructions, a detected variance in the measurements, or another metric. Then, at operation 1016, in subsequent repeats of the test, the system may compare biometric measurements of the user to the baseline measurement to determine deviations. The system may detect an increase in force/pressure at a tactile threshold as indicating peripheral neuropathy. The system may generate an output indicating a condition of peripheral neuropathy, such as a presence, absence, or level/scoring.

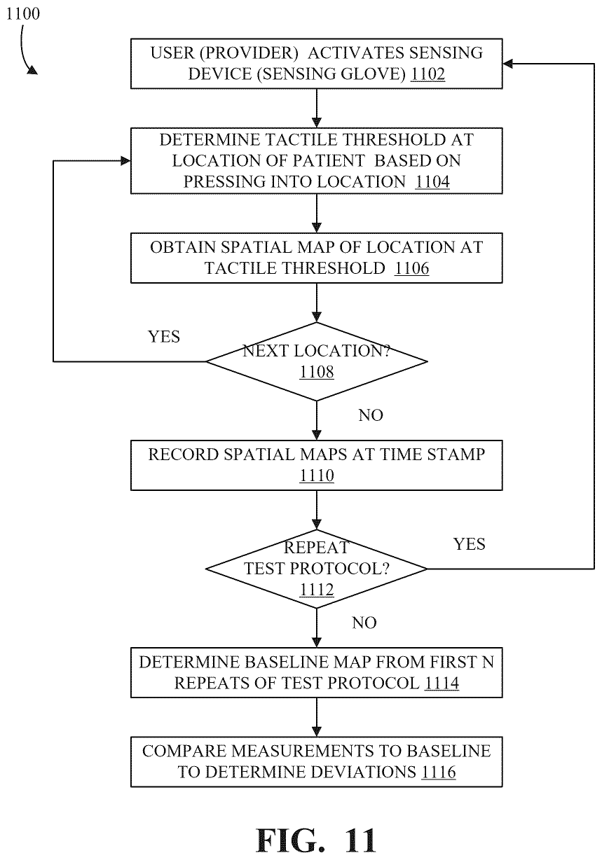

FIG. 11 is an example of a second process 1100 (a test protocol, or test) for determining peripheral neuropathy of an individual, utilizing the sensing device 100. At operation 1102, a user (e.g., a health provider) activates the sensing device 100 (e.g., a sensing glove), such as by donning the sensing device 100. For example, the sensing device 100 may include the sensor array 114A outward facing to detect forces at an exterior surface of the sensing device toward a patient in the environment. At operation 1104, the system may determine a tactile threshold for a location (e.g., a finger, fingertip, thumb, thumb tip, toe, etc.) of a patient based on the user pressing the patient at a specific locations, gradually increasing applied force until the patient can feel the applied force and provide feedback (e.g., detected by the system as a cue from the user or patient, such as via the microphone 136). At operation 1106, the system may obtain a biometric measurement to produce/record a spatial map of the location that corresponds to the tactile threshold (e.g. a force or pressure map). At operation 1108, the user may repeat the measurement for a next location. If repeating the measurements (Yes), the system may return to operation 1104 for the next location. However, if not repeating the measurement (No), at operation 1110 the system may record spatial maps for each of the location (e.g., five digits per hand, 10 digits total, etc.) corresponding to a time stamp, such as a particular date and time. At operation 1112, the user may repeat the test periodically, one or more times over the course of weeks, months, or years. In some cases, the system may generate an alert to the user as a reminder to repeat the test. If the user repeats the test (Yes), the test may be performed again beginning at operation 1102. However, if the user does not repeat the test (No), at operation 1114 the system may determine a baseline measurement (e.g., a baseline map of reference values) for the patient, from the first N repeats of the test, where N is an integer greater than or equal to one. In some cases, N may be determined by health provider's instructions, a detected variance in the measurements, or another metric. Then, at operation 1116, in subsequent repeats of the test, the system may compare biometric measurements of the patient to the baseline measurement to determine deviations. The system may detect an increase in force/pressure at a tactile threshold as indicating peripheral neuropathy. The system may generate an output indicating a condition of peripheral neuropathy, such as a presence, absence, or level/scoring.

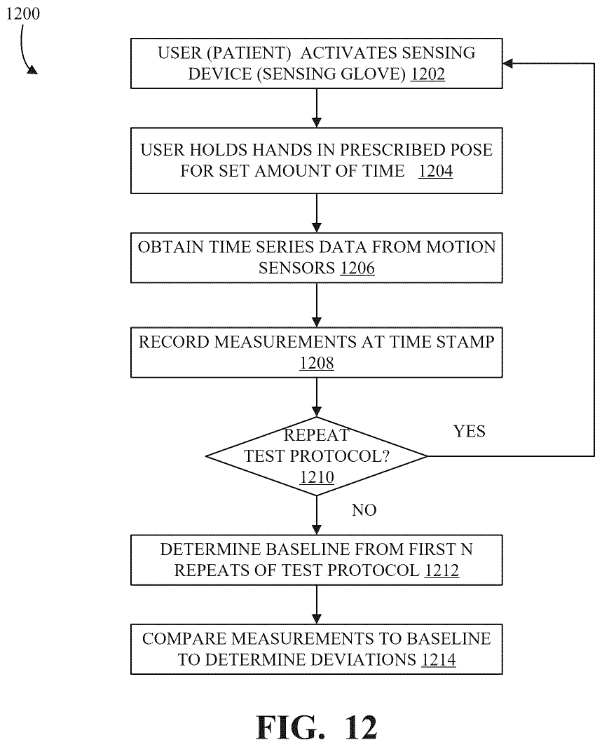

FIG. 12 is an example of a process 1200 (a test protocol, or test) for determining Parkinson's disease of an individual, utilizing the sensing device 100. At operation 1202, a user (e.g., a patient) activates the sensing device 100 (e.g., a sensing glove), such as by donning the sensing device 100. The sensing device 100 may include motion sensors (e.g., the motion sensors 116), accelerometers, gyroscopes, and/or magnetometers to detect finger tremors. At operation 1204, the user may hold the sensing device 100 in a prescribed pose for a set amount of time (e.g. a hand, wearing the sensing glove, out in front of the patient's body for one minute). In this time, the patient attempts to keep the location (fingers/hands) as stable as possible. At operation 1206, the system may obtain a biometric measurement of the location, such as time series data from the motion sensors. The system can combine the time series data to quantify location stability (e.g. finger/hand stability, quantified by a standard deviation of linear acceleration in each of three axes). At operation 1208, the system may record the measurements (e.g., collectively biometric measurements, including raw data and calculated metrics) corresponding to a time stamp, such as a particular date and time. At operation 1210, the user may repeat the test periodically, one or more times over the course of weeks, months, or years. In some cases, the system may generate an alert to the user as a reminder to repeat the test, which may be configured at the direction of a health provider. If the user repeats the test (Yes), the test may be performed again beginning at operation 1202. However, if the user does not repeat the test (No), at operation 1212 the system may determine a baseline measurement (e.g., a baseline map of reference values) for the user, from the first N repeats of the test, where N is an integer greater than or equal to one. In some cases, N may be determined by the health provider's instructions, a detected variance in the measurements, or another metric. Then, at operation 1214, in subsequent repeats of the test, the system may compare biometric measurements of the user to the baseline measurement to determine finger tremors. The system may detect an increase in tremor magnitude at a location (e.g., finger/hand) as indicating a progression of Parkinson's disease. The system may generate an output indicating a condition of Parkinson's disease, such as a presence, absence, or level/scoring.

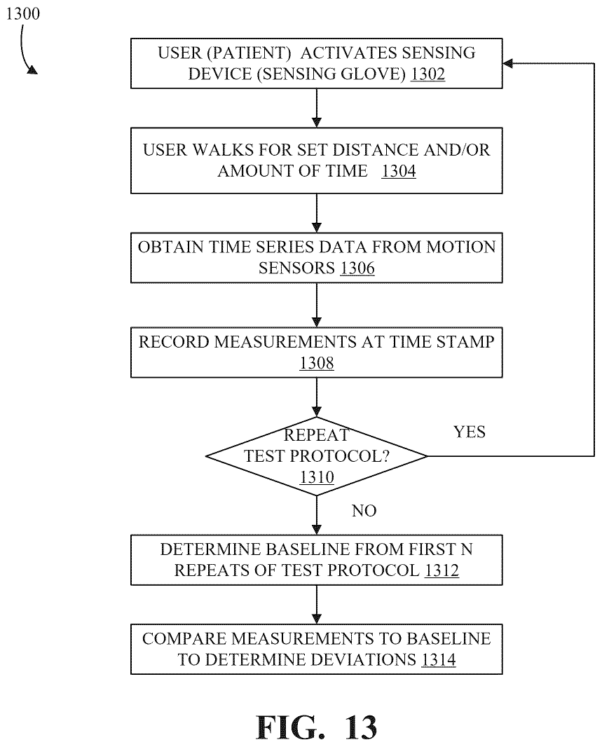

FIG. 13 is an example of a process 1300 (a test protocol, or test) for determining dementia of an individual, utilizing the sensing device 100. At operation 1302, a user (e.g., a patient) activates the sensing device 100 (e.g., a sensing glove), such as by donning the sensing device 100. The sensing device 100 may include motion sensors (e.g., the motion sensors 116 and/or 116W), accelerometers, gyroscopes, and/or magnetometers to detect a gait of the user. At operation 1304, the user may walk with the sensing device 100 for a set distance or amount of time (e.g. walking, while wearing the sensing glove, for one minute or 100 feet). At operation 1306, the system may obtain a biometric measurement of the user, such as time series data from the motions sensors. The system can combine the time series data to quantify walking stability, consistency, and/or asymmetry (e.g. stride length, double-support time, speed, left/right arm swing). At operation 1308, the system may record the measurements (e.g., collectively biometric measurements, including raw data and calculated metrics) corresponding to a time stamp, such as a particular date and time. At operation 1310, the user may repeat the test periodically, one or more times over the course of weeks, months, or years. In some cases, the system may generate an alert to the user as a reminder to repeat the test, which may be configured at the direction of a health provider. If the user repeats the test (Yes), the test may be performed again beginning at operation 1302. However, if the user does not repeat the test (No), at operation 1312 the system may determine a baseline measurement (e.g., a baseline map of reference values) for the user, from the first N repeats of the test, where N is an integer greater than or equal to one. In some cases, N may be determined by the health provider's instructions, a detected variance in the measurements, or another metric. Then, at operation 1314, in subsequent repeats of the test, the system may compare biometric measurements of the user to the baseline measurement to determine a significant decrease in walking stability (e.g. increase in double-support time) or unstable gait as indicating a progression of dementia. The system may generate an output indicating a condition of dementia, such as a presence, absence, or level/scoring.

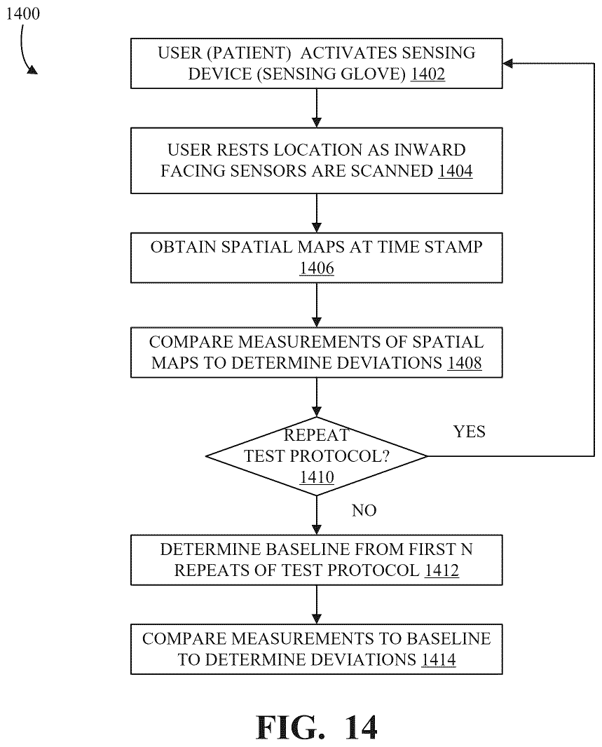

FIG. 14 is an example of a first process 1400 (a test protocol, or test) for determining a blood clot of an individual, utilizing the sensing device 100. At operation 1402, a user (e.g., a patient) activates the sensing device 100 (e.g., a sensing glove), such as by donning the sensing device 100. For example, the sensing device 100 may include the sensor array 114B inward facing to detect forces at an interior surface of the sensing device. At operation 1404, the user may rest with the sensing device 100 (e.g., rest their hand on a table) as the sensor array 114B scans the user. At operation 1406, the system may obtain a biometric measurement to produce/record a spatial map of blood pressure of the location. The system may record a spatial map corresponding to a time stamp, such as a particular date and time. At operation 1408, the system may compare biometric measurements (e.g., spatial maps) to one another to identify deviations in blood pressure exceeding a threshold within each spatial map. A significant deviation may indicate a blood clot. At operation 1410, the user may repeat the test periodically, one or more times over the course of weeks, months, or years. In some cases, the system may generate an alert to the user as a reminder to repeat the test, which may be configured at the direction of a health provider. If the user repeats the test (Yes), the test may be performed again beginning at operation 1402. However, if the user does not repeat the test (No), at operation 1412 the system may determine a baseline measurement (e.g., a baseline map of reference values) for the user, from the first N repeats of the test, where N is an integer greater than or equal to one. In some cases, N may be determined by the health provider's instructions, a detected variance in the measurements, or another metric. Then, at operation 1414, in subsequent repeats of the test, the system may compare biometric measurements of the user to the baseline measurement to determine deviations. The system may detect deviations exceeding a threshold as indicating development of a blood clot. The system may generate an output indicating the blood clot, such as a presence, absence, or level/scoring.

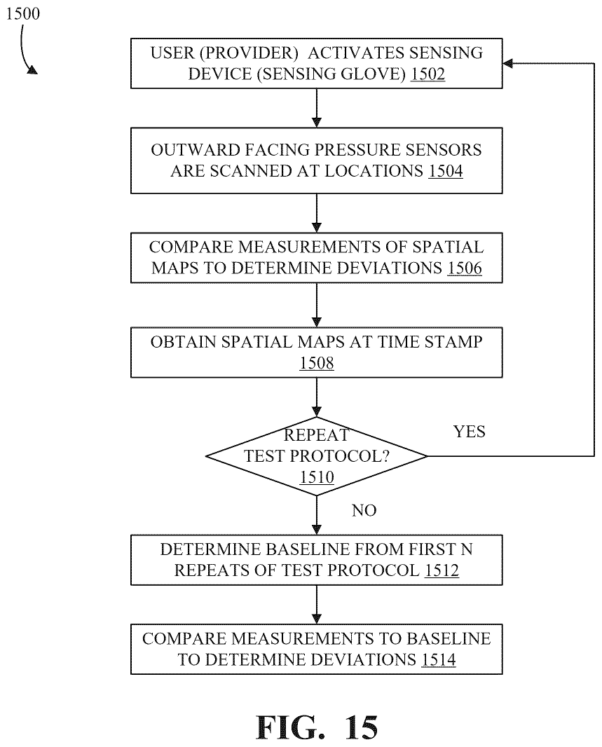

FIG. 15 is an example of a second process 1500 (a test protocol, or test) for determining a blood clot of an individual, utilizing the sensing device 100. At operation 1502, a user (e.g., a health provider) activates the sensing device 100 (e.g., a sensing glove), such as by donning the sensing device 100. For example, the sensing device 100 may include the sensor array 114A outward facing to detect forces at an exterior surface of the sensing device toward a patient in the environment. At operation 1504, the user may rest the sensing device 100 on a location of a patient where a blood clot may be suspected as the sensor array 114A scans the patient. At operation 1506, the system may obtain a biometric measurement to produce/record a spatial map of blood pressure of the location. The system may record a spatial map corresponding to a time stamp, such as a particular date and time. At operation 1508, the system may compare biometric measurements (e.g., spatial maps) to one another to identify deviations in blood pressure exceeding a threshold within each spatial map. A significant deviation may indicate a blood clot. At operation 1510, the user may repeat the test periodically, one or more times over the course of weeks, months, or years. In some cases, the system may generate an alert to the user as a reminder to repeat the test, which may be configured at the direction of a health provider. If the user repeats the test (Yes), the test may be performed again beginning at operation 1502. However, if the user does not repeat the test (No), at operation 1512 the system may determine a baseline measurement (e.g., a baseline map of reference values) for the patient, from the first N repeats of the test, where N is an integer greater than or equal to one. In some cases, N may be determined by the health provider's instructions, a detected variance in the measurements, or another metric. Then, at operation 1514, in subsequent repeats of the test, the system may compare biometric measurements of the patient to the baseline measurement to determine deviations. The system may detect deviations exceeding a threshold as indicating development of a blood clot. The system may generate an output indicating the blood clot, such as a presence, absence, or level/scoring.

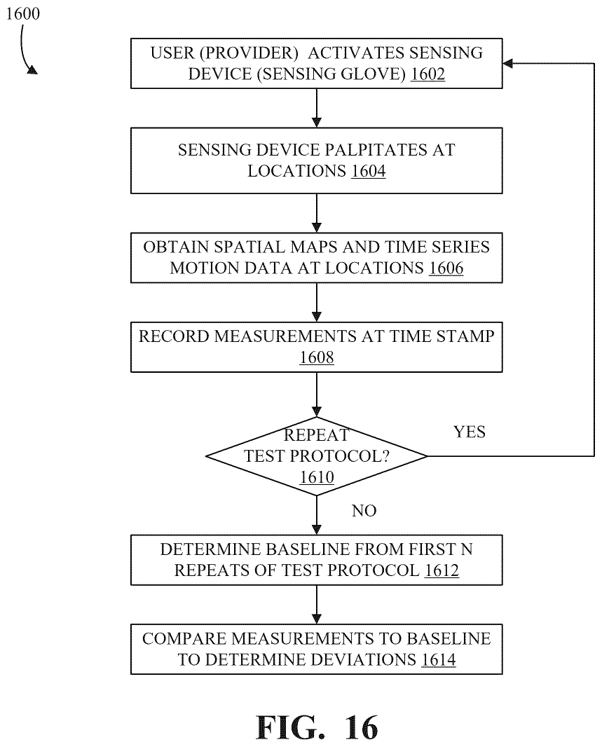

FIG. 16 is an example of a second process 1600 (a test protocol, or test) for determining a tumor of an individual, utilizing the sensing device 100. At operation 1602, a user (e.g., a health provider) activates the sensing device 100 (e.g., a sensing glove), such as by donning the sensing device 100. For example, the sensing device 100 may include the sensor array 114A outward facing to detect forces at an exterior surface of the sensing device toward a patient in the environment. The sensing device 100 may also include motion sensors (e.g., the motion sensors 116 and/or 116W), accelerometers, gyroscopes, and/or magnetometers. At operation 1604, the user may utilize the sensing device 100 to palpitate a location of a patient where a tumor may be suspected (e.g., breast tissue). At operation 1606, the system may obtain a biometric measurement to produce/record spatial maps of pressure and temperature and time series data from the motions sensors during examination at the location. The system may record a spatial map corresponding to a time stamp, such as a particular date and time. The system can combine the pressure, temperature, and/or time series data to produce a map of tissue stiffness (e.g., measured pressure is proportional to displacement during palpitation, as measured by the motion sensors, multiplied by a tissue stiffness). The system can identify deviations in tissue stiffness or temperature within the spatial maps that exceed a threshold, which may be an indication of a tumor. At operation 1608, the system may record the spatial maps and motions measurements (e.g., collectively biometric measurements, including raw data and calculated metrics) corresponding to a time stamp, such as a particular date and time. At operation 1610, the user may repeat the test periodically, one or more times over the course of weeks, months, or years. In some cases, the system may generate an alert to the user as a reminder to repeat the test, which may be configured at the direction of a health provider. If the user repeats the test (Yes), the test may be performed again beginning at operation 1602. However, if the user does not repeat the test (No), at operation 1612 the system may determine a baseline measurement (e.g., a baseline map of reference values) for the patient, from the first N repeats of the test, where N is an integer greater than or equal to one. In some cases, N may be determined by the health provider's instructions, a detected variance in the measurements, or another metric. Then, at operation 1614, in subsequent repeats of the test, the system may compare biometric measurements of the patient to the baseline measurement to determine deviations. The system may detect deviations exceeding a threshold as indicating tumor growth. The system may generate an output indicating the tumor, such as a presence, absence, or level/scoring.

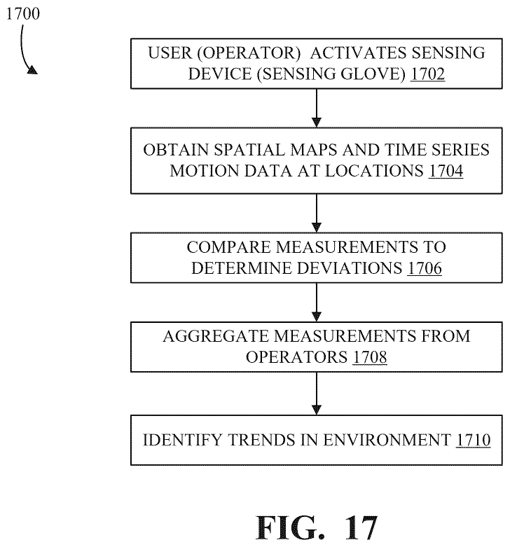

FIG. 17 is an example of a process 1700 (a test protocol, or test) for monitoring safety or ergonomics of an individual, utilizing the sensing device 100 to perform a task. At operation 1702, a user (e.g., an operator) activates the sensing device 100 (e.g., a sensing glove), such as by donning the sensing device 100. For example, the sensing device 100 may include the sensor array 114B inward facing to detect forces at an interior surface of the sensing device. At operation 1704, the system may obtain a biometric measurement to produce/record spatial maps of pressure and temperature and time series data from the motions sensors during examination at the location. The system may record a spatial map corresponding to a time stamp, such as a particular date and time. The system may record the spatial maps and motions measurements (e.g., collectively biometric measurements, including raw data and calculated metrics) corresponding to a time stamp, such as a particular date and time. The system may obtain and record the biometric measurements during a work shift, such as periodically during a multi-hour shift, multiple days of a week, or multiple weeks of a month. At operation 1706, the system may determine, in real time with performance of the task, a baseline measurement (e.g., a baseline map of reference values) for the user and compare biometric measurements of the user to the baseline measurement to determine deviations. For example, the system can identify to the user one or more unsafe or non-ergonomic conditions, such as a one-time pressure exceeding a prescribed threshold, pressure repeatedly exceeding a lower threshold, a same task repeated too many times in a given interval (potentially leading to repetitive strain injury), etc. The system may generate an output indicating the unsafe or non-ergonomic, such as a presence, absence, or level/scoring of the behavior. In some cases, feedback to the user may be provided via the LED 135, a display, a speaker (e.g., an audible buzzer), haptic feedback (e.g., driven via the sensor array 114B or other piezoelectric sensors), or other user interface element, integrated with sensing device 100. The feedback may enable the user to modify unsafe or non-ergonomic behavior or take a break to reduce risk of injury. At operation 1708, the system may aggregate data from one or more other users (e.g., other operators perform the task) in a central database. At operation 1710, over multiple shifts, the system may identify trends across the entire environment (e.g., a factory workforce) including operators or tasks with the highest risk of injury. This information may enable a supervisor system to implement interventions including re-training, modifications to standard operating procedures, additional breaks or recovery time, and/or elimination of certain tasks.

An aspect of the disclosure may include a non-transitory machine-readable medium (such as computer memory) having stored thereon instructions, which program one or more data processing components (generically referred to here as a “processor”) to (automatically) perform operations, as described herein. In other aspects, some of these operations might be performed by specific hardware components that contain hardwired logic. Those operations might alternatively be performed by any combination of programmed data processing components and fixed hardwired circuit components. A “processor” may include a distributed arrangement where multiple processors are configured and controlled to perform the recited operations or tasks together, e.g., one processor can perform some of the recited operations and another processor can perform others of the recited operations.

As used herein, the term “circuitry” refers to an arrangement of electronic components (e.g., transistors, resistors, capacitors, and/or inductors) that is structured to implement one or more functions. For example, a circuit may include one or more transistors interconnected to form logic gates that collectively implement a logical function.

In utilizing the various aspects of the embodiments, it would become apparent to one skilled in the art that combinations or variations of the above embodiments are possible for utilizing sensing gloves to perform tasks. Although the embodiments have been described in language specific to structural features and/or methodological acts, it is to be understood that the appended claims are not necessarily limited to the specific features or acts described. The specific features and acts disclosed are instead to be understood as embodiments of the claims useful for illustration.

1. A system for biometric measurements, comprising:
a wearable article to be worn by a user;
a sensor array coupled to the wearable article, the sensor array including sensors that are submillimeter in at least one in-plane dimension and are distributed at multiple locations of a sensing area of the wearable article; and
a processor configured to:
obtain a biometric measurement from the sensor array, the biometric measurement including a spatial map of sensed conditions from the sensors at the multiple locations; and
generate an output based on comparing the spatial map to a baseline map of reference values corresponding to the multiple locations.

2. The system of claim 1, wherein the output is generated based on determining deviations between the sensed conditions and the reference values.

3. The system of claim 1, wherein the output includes an alert based on the sensed conditions differing from the reference values by more than a threshold.

4. The system of claim 1, wherein the output confirms an identity of the user based on the sensed conditions matching the reference values to within a threshold.

5. The system of claim 1, wherein the output enables performance of equipment to be linked to the user.

6. The system of claim 1, wherein the biometric measurement indicates a blood oxygen level, heart rate, blood pressure, blood flow, body temperature, or skin conductivity.

7. The system of claim 1, wherein the sensor array is inward facing within the wearable article to enable the sensor array to contact the user wearing the wearable article.

8. The system of claim 1, wherein the sensor array is outward facing from the wearable article to enable the sensor array to contact a surrounding environment.

9. The system of claim 1, wherein the sensor array is outward facing from the wearable article, and further comprising another sensor array that is inward facing, wherein the biometric measurement of one sensor array is compensated by another.

10. The system of claim 1, further comprising:
a motion sensor coupled to the wearable article, wherein the processor further executes to obtain a gait measurement of the user from the motion sensor.

11. The system of claim 1, further comprising:
a plurality of motion sensors, each motion sensor coupled to a digit of the wearable article, wherein the processor further executes to obtain measurements of the user from the plurality of motion sensors.

12. The system of claim 1, further comprising:
a power source supplying power to the wearable article, wherein the processor further executes to:
select either 1) a coarse scan in which fewer sensors of the sensor array are utilized to conserve the power source, or 2) a fine scan in which more sensors of the sensor array are utilized to increase resolution of the spatial map.

13. The system of claim 1, wherein the wearable article comprises a glove with the sensor array arranged on a digit of the glove.

14. The system of claim 1, wherein the wearable article comprises a sleeve to be worn on an arm or leg of the user.

15. The system of claim 1, wherein the sensors are piezoelectric sensors that are less than 2 millimeters (mm) apart.

16. A method for biometric measurements, comprising:
obtaining a biometric measurement from a sensing device comprising a sensor array coupled to a wearable article, wherein the sensor array includes sensors that are submillimeter in at least one in-plane dimension and are distributed at multiple locations of a sensing area of the wearable article, and wherein the biometric measurement includes a spatial map of sensed conditions from the sensors at the multiple locations; and
generating an output based on comparing the spatial map to a baseline map of reference values corresponding to the multiple locations.

17. The method of claim 16, wherein the output includes an alert based on the sensed conditions differing from the reference values by more than a threshold.

18. The method of claim 16, further comprising:
rejecting an identity of a user based on the output indicating the sensed conditions differing from the reference values by more than a threshold.

19. The method of claim 16, further comprising:
confirming an identity of a user based on the output indicating the sensed conditions matching the reference values to within a threshold.

20. The method of claim 16, further comprising:
enabling performance of the sensing device to be linked to a user.

21. The method of claim 16, further comprising:
operating a plurality of sensors of the sensor array in a first mode during a first time, then operating the plurality of sensors in a second mode during a second time.

22. The method of claim 16, further comprising:
generating acoustic energy from the sensor array, then obtaining the biometric measurement in response to the acoustic energy.

23. The method of claim 16, wherein a plurality of sensor arrays coupled to the wearable article operate synchronously with one another to form a large-area sensing device.

24. The method of claim 16, wherein the biometric measurement is responsive to a self-check performed by a user.

25. The method of claim 16, wherein the baseline map is generated from an initial biometric measurement.

26. The method of claim 16, wherein the output indicates a condition of peripheral neuropathy of a user wearing the sensing device based on the sensor array being inward facing to detect forces at an interior surface of the sensing device.

27. The method of claim 16, wherein the output indicates a condition of peripheral neuropathy of a patient in an environment of the sensing device based on the sensor array being outward facing to detect forces at an exterior surface of the sensing device.

28. The method of claim 16, wherein the output indicates a condition of Parkinson's disease of a user wearing the sensing device based on a motion sensor coupled to the wearable article detecting finger tremors.

29. The method of claim 16, wherein the output indicates a condition of dementia of a user wearing the sensing device based on a motion sensor coupled to the wearable article detecting an unstable gait.

30. The method of claim 16, wherein the output indicates a blood clot of a user wearing the sensing device based on the sensor array being inward facing to detect forces at an interior surface of the sensing device.

31. The method of claim 16, wherein the output indicates a blood clot of a patient in an environment of the sensing device based on the sensor array being outward facing to detect forces at an exterior surface of the sensing device.

32. The method of claim 16, wherein the output indicates a tumor of a patient in an environment of the sensing device based on palpating a location of the patient and the sensor array being outward facing to detect forces at an exterior surface of the sensing device.

33. The method of claim 16, wherein the output indicates exceeding a safety or ergonomic condition by a user wearing the sensing device based on comparing to the baseline map of one or more other users.

34. The method of claim 16, further comprising:
obtaining biometric measurements periodically during a multi-hour shift, multiple days of a week, or multiple weeks of a month.
